(19) United States
(12) Patent Application Publication
(10) Pub. No.: US 2026/0225062 A1
(43) Pub. Date: Aug. 6, 2026

(54) CONDITION-EXPLORATION MICROREACTOR SYSTEM AND PROCESS CONDITION OPTIMIZATION METHOD

(71) Applicant: Hitachi Plant Services Co., Ltd., Tokyo (JP)

(72) Inventors: Yukako ASANO, Tokyo (JP); Masashi TAGAMI, Tokyo (JP); Takehiro CHIBA, Tokyo (JP)

(21) Appl. No.: 19/149,946

(22) PCT Filed: Dec. 26, 2023
(86) PCT No.: PCT/JP2023/046731

§ 371 Date: Jul. 22, 2025

(30) Foreign Application Priority Data
Feb. 13, 2023 (JP) 2023-019798

(51) Int. Cl. B01J 19/00; B01J 13/02

(52) U.S. Cl. CPC B01J 19/0093; B01J 13/02; B01J 19/0033; B01J 2219/00202; B01J 2219/00243; B01J 2219/00889; B01J 2219/00957; B01J 2219/00986

(57) ABSTRACT

The condition-exploration microreactor system includes: a microreactor having two inlets from which fluids are introduced and a flow path where the fluids merge, the microreactor mixing, in the flow path, a first raw material introduced from a first inlet of the two inlets and a second raw material introduced from a second inlet of the two inlets; a first raw material container in which the first raw material is prepared; a second raw material container in which the second raw material is prepared; a product collection container that collects a product obtained by mixing the first raw material and the second raw material in the microreactor; a first liquid delivery pump that delivers the first raw material in the first raw material container to the first inlet; and a second liquid delivery pump that delivers the second raw material in the second raw material container to the second inlet.

### TECHNICAL FIELD

The present invention relates to microreactors for mixing raw materials.

### BACKGROUND ART

In recent years, the use of microreactors has been advancing in the fields of the production of bio-related products, drugs, chemical products, and the like. Microreactors are flow-type reactors having micro flow paths on the order of micrometers, and are used for mixing of fluids or for reactions between fluids. Microreactors are typically fabricated using microprocessing technologies such as molding and lithography, and detachable models of micrometers capable of replacement and single-use models of micrometers assuming disposable have been examined.

In microreactors, micro flow paths are used as reaction fields, and accordingly, fluids can be mixed rapidly by molecular diffusion. In addition, compared with conventional batch methods using large-sized reactors, microreactors have a relatively significant surface area effect relative to fluid volume, and accordingly, have a merit of enhanced efficiency in heat transfer, heat conduction, chemical reactions, and the like. Accordingly, there is a possibility that, even reactions that pose a risk of undesirable runaway due to heat generation in typical batch reactions, reactions that require precise temperature control, and reactions that require rapid heating or cooling can be performed easily using microreactors.

Furthermore, the effect of flow path widths relative to liquid delivery flow rates is relatively significant in micro flow paths as compared with the batch methods, and accordingly, even at the same Reynolds numbers as the batch methods, the shear rates become higher in micro flow paths. In addition, the droplet size is decided by operation conditions such as the flow rates of fluids, in addition to the sizes of flow paths. Accordingly, there is a possibility that, even emulsification that requires uniformity and particle size control, nanoparticle generation that requires uniformity and particle size control, antisolvent crystallization, and the like can be performed easily using microreactors.

Due to such characteristics, shortening of reaction times and enhancement of reaction yields (generation efficiency) through application of microreactors are expected in various fields.

Meanwhile, in a case where microreactors are applied to reactions, similarly to the batch methods, it is necessary to optimize reaction conditions such as reaction temperature and reaction time. As known reaction condition optimization methods, there are the design of experiments in which experimental conditions to be examined are determined from factors and levels and optimum reaction conditions are explored, and reaction rate analysis in which reactions are decomposed into elementary reactions, the reaction rate constant for each elementary reaction is determined, and optimum reaction conditions are predicted. Various examinations have been conducted on reaction rate analysis using experimental results obtained from the batch methods.

For example, in Patent Document 1, as setting of a reaction model in which the constituent concentration of an exhaust gas changes due to contact with a catalyst, diffusion and chemical reaction rate equations of this model are created, and then, these equations are solved to determine a characteristic formula representing changes in the constituent concentration. According to the description of Patent Document 1, a rig test is performed under a predetermined condition to determine changes in the constituent concentration, the values of activation energy, frequency factor, and diffusion coefficient in the characteristic formula are changed to explore values that can reproduce rig test results, and, when rig test results can be reproduced, the reaction model is determined as appropriate, and the coefficients of temperature/concentration characteristics are decided.

In addition, in Patent Document 2, in a process in which a polymerization reaction is caused using a batch reactor, a simulating section simulates product quality at constant quality using process data measured from the process and a process model, and corrects the process model. A prediction determining section determines whether or not the predicted product quality falls outside a tolerance quality range, and, in a case where the predicted product quality falls outside the tolerance quality range, a correction operation section predicts execution results of a correction operation on the process by simulation, and decides the correction operation and an optimum operation amount thereof. Then, according to the description of Patent Document 2, the decided correction operation and optimum operation amount are output to a general-purpose system, and polymerization reactions of the process are controlled.

In addition, Patent Document 3 describes a method and an apparatus for evaluating a catalytic converter that processes an exhaust gas. A flow reaction section in which a flow path is filled with a reaction member, which flow reaction section forms a reaction apparatus, measures the reaction efficiency of the reaction member under conditions of fluid composition, flow rate, temperature, and pressure, and a reaction modelling section forms a reaction model of the reaction member using reaction efficiency data and extracts a reaction rate constant representing dependency on temperature and gas constituent concentrations. An efficiency predicting/detecting section calculates and predicts the efficiency of the reaction apparatus using the reaction model, the physical properties of the reaction member and a reaction apparatus material, and the specifications of the reaction apparatus, and an optimization evaluating section selects optimum reaction apparatus specifications and an optimum reaction member and reaction apparatus on the basis of the correspondence between results of the efficiency prediction and the original use conditions of the reaction apparatus.

In addition, Patent Document 4 discloses reaction rate analysis using experimental results of microreactors, and describes a reaction rate constant measuring apparatus having: microreactors including micro flow paths where a plurality of fluids are caused to merge, and different types of substance included in the respective fluids are caused to react with each other; and analysis means that quantifies reaction products generated by the reactions in the microreactors. A plurality of the microreactors whose micro flow paths have mutually different diffusion distances are provided, the analysis means quantifies a reaction product generated in each microreactor to output the quantitative results, and reaction rate constants of different types of substance are calculated on the basis of a plurality of the quantitative results sent from the analysis means.

### PRIOR ART DOCUMENT

### Patent Documents

Patent Document 1: JP-2004-8908-A

Patent Document 2: JP-2001-106703-A

Patent Document 3: Japanese Patent No. 3376843

Patent Document 4: Japanese Patent No. 4701190

### SUMMARY OF THE INVENTION

### Problem to be Solved by the Invention

Meanwhile, in optimization of reaction conditions based on the design of experiments, it is possible to reduce the number of experiments while taking the magnitude of interactions between factors into consideration from all patterns of combinations of factors and levels using orthogonal arrays, and obtain the effects of each factor.

However, determining the magnitude of interactions between factors requires experience, and basically, the number of experiments equivalent to the power (NP) of the number N of levels is required. In addition, in experiments using orthogonal arrays, factors lack continuity, and accordingly, there is a problem that values cannot be predicted for factors that have not been experimented. As the number of experiments required to optimize reaction conditions increases, the time for experiments increases, and the amount of reagents to be used increases, leading to higher costs and more waste.

In addition, depending on the types of reactions, there may be a case where intermediate products that cannot be measured with an analyzing apparatus are generated, or a case where, although intermediate products may be measured with an analyzing apparatus, the intermediate products cannot be effectively measured because the analysis speed cannot keep up due to the short lifetimes of the intermediate products. Furthermore, in a case where a plurality of raw materials or products are taken into consideration, there is a possibility that inconsistencies in analysis timing are generated undesirably due to the use of multiple types of analyzing apparatus, and experimental results are affected. Accordingly, there is a possibility that accurate evaluation cannot be performed in reaction rate analysis.

Such problems are not examined sufficiently in the technologies of Patent Documents 1 to 4 described above, and there is room for improvement in terms of analysis costs and reliability.

In view of this, an object of the present invention is to provide a condition-exploration microreactor system and a process condition optimization method being excellent in terms of cost and reliability, which make it possible to predict values for factors that have not been experimented or factors that cannot be measured, while reducing the number of experiments required to optimize conditions of processes such as reactions as much as possible.

### Means for Solving the Problem

In order to solve the problem, the present invention provides a condition-exploration microreactor system including: a microreactor having two inlets from which fluids are introduced and a flow path where the fluids are caused to merge, the microreactor mixing, in the flow path, a first raw material introduced from a first inlet of the two inlets and a second raw material introduced from a second inlet of the two inlets; a first raw material container in which the first raw material is prepared; a second raw material container in which the second raw material is prepared; a product collection container that collects a product obtained by mixing the first raw material and the second raw material in the microreactor; a first liquid delivery pump that delivers the first raw material in the first raw material container to the first inlet; a second liquid delivery pump that delivers the second raw material in the second raw material container to the second inlet; a control section that operates the first liquid delivery pump and the second liquid delivery pump; an analyzing section that analyzes the product; and an operation/analysis section that receives an input of a signal from and outputs a signal to the control section and the analyzing section, and analyzes optimization of a process condition, in which a process model for a process in which the product is generated from the first raw material and the second raw material is constructed, a process rate constant in the process model is calculated, and a next experimental condition is decided on the basis of time-series data calculated from the process rate constant.

In addition, the present invention provides a process condition optimization method of optimizing process conditions of a microreactor, the process condition optimization method including: (a) a step of constructing a process model for a process in which a product is generated from a first raw material and a second raw material; (b) a step of implementing the process in which the product is generated from the first raw material and the second raw material under a predetermined condition; (c) a step of analyzing the product generated at the step (b), and acquiring experiment data; (d) a step of calculating a process rate constant in the process model constructed at the step (a) by using the experiment data acquired at the step (c); (e) a step of calculating time-series data from the process rate constant calculated at the step (d); and (f) a step of deciding a next experimental condition on the basis of the time series data calculated at the step (e).

### Advantages of the Invention

According to the present invention, it is possible to realize a condition-exploration microreactor system and a process condition optimization method that make it possible to predict values for factors that have not been experimented or factors that cannot be measured, while reducing the number of experiments required to optimize process conditions such as reactions as much as possible, the condition-exploration microreactor system and the process condition optimization method being excellent in terms of cost and reliability.

Thereby, it is possible to reduce the time required for experiments and the amount of reagents (waste) required for the experiments.

Problems, configurations, and advantages other than those described above are made clear by the following explanation of embodiments.

### BRIEF DESCRIPTION OF THE DRAWINGS

FIG. 1 is a schematic diagram of a condition-exploration microreactor system according to a first embodiment of the present invention.

FIG. 2 is a figure depicting an example of a microreactor.

FIG. 3 is a flowchart depicting a process condition optimization method according to the first embodiment of the present invention.

FIG. 4 is a schematic diagram of a condition-exploration microreactor system according to a second embodiment of the present invention.

FIG. 5 is a schematic diagram of the condition-exploration microreactor system according to a third embodiment of the present invention.

FIG. 6 is a schematic diagram of the condition-exploration microreactor system according to a fourth embodiment of the present invention.

### MODES FOR CARRYING OUT THE INVENTION

Hereinbelow, embodiments of the present invention are explained using the figures. Note that identical configurations are given identical reference signs in the respective figures, and detailed explanations of overlapping portions are omitted.

### First Embodiment

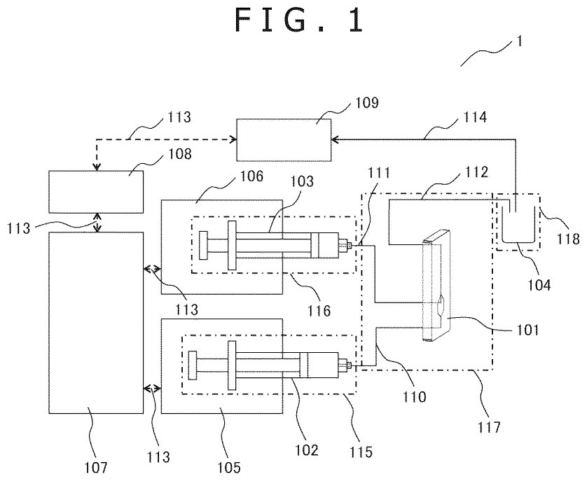

A condition-exploration microreactor system and a process condition optimization method according to a first embodiment of the present invention are explained with reference to FIG. 1 to FIG. 3.

FIG. 1 is a schematic diagram of a condition-exploration microreactor system 1 according to the present embodiment.

As depicted in FIG. 1, the condition-exploration microreactor system 1 according to the present embodiment includes, as main constituent elements, a microreactor 101, a first raw material container 102, a second raw material container 103, a product collection container 104, a first liquid delivery pump 105, a second liquid delivery pump 106, a control section 107, an operation/analysis section 108, an analyzing section 109, a first connecting section 110, a second connecting section 111, a residence section 112, signal lines 113, sampling for analysis 114, a temperature adjusting apparatus 115, a temperature adjusting apparatus 116, a temperature adjusting apparatus 117, and a temperature adjusting apparatus 118.

In addition, although not illustrated, the residence section 112: may include a tube and a fitting connecting the tube and the microreactor 101; may include a device in which a residence flow path is formed and a fitting connecting the device and the microreactor 101; may include a tube, a fitting connecting the tube and the microreactor 101, and a fitting connecting the tube and the product collection container 104; may include a device in which a residence flow path is formed, a fitting connecting the device and the microreactor 101 and a fitting connecting the device and the product collection container 104; or may include a fitting directly connecting the product collection container 104 and the microreactor 101, and the like.

Note that, in a case where functions of the residence section 112 are not required, e. g., in a case where the process in the microreactor 101 ends instantaneously, the product is collected directly from the microreactor 101 into the product collection container 104 without using a fitting or the like. In particular, in a case where the process in the microreactor 101 is particle generation, an excessively long residence section increases the risk of blockage in the residence section, and accordingly, a configuration in which the residence section 112 is not provided, and the microreactor 101 and the product collection container 104 are directly connected is preferable.

The first connecting section 110 may include a tube, a fitting connecting the tube and the first raw material container 102, and a fitting connecting the tube and the microreactor 101, or may include a fitting directly connecting the first raw material container 102 and the microreactor 101, and the like. In addition, the second connecting section 111 may include a tube, a fitting connecting the tube and the second raw material container 103, and a fitting connecting the tube and the microreactor 101, or may include a fitting directly connecting the second raw material container 103 and the microreactor 101, and the like.

The temperature adjusting apparatus 115, the temperature adjusting apparatus 116, the temperature adjusting apparatus 117, and the temperature adjusting apparatus 118 are provided to adjust, to predetermined temperatures, predetermined areas in the system as represented by broken lines in the figure. The first raw material container 102 is included in the adjustment range of the temperature adjusting apparatus 115. The second raw material container 103 is included in the adjustment range of the temperature adjusting apparatus 116. The first connecting section 110, the second connecting section 111, the microreactor 101, and the residence section 112 are included in the adjustment range of the temperature adjusting apparatus 117. The product collection container 104 is included in the adjustment range of the temperature adjusting apparatus 118.

The microreactor 101 is a flow-type reactor, and has: two inlets from which individual fluids are introduced from the outside; micro flow paths where the introduced fluids are caused to merge; and an outlet from which a product fluid generated by the merging flows out to the outside. The microreactor 101 mixes, in the micro flow paths, a fluid introduced from a first inlet and a fluid introduced from a second inlet. By mixing the fluids, a product fluid that has initiated a predetermined process is generated.

A first raw material is prepared in the first raw material container 102. The first inlet of the microreactor 101 is connected with the first raw material container 102 via the first connecting section 110. The first liquid delivery pump 105 delivers the first raw material from the first raw material container 102 to the first inlet of the microreactor 101.

A second raw material is prepared in the second raw material container 103. The second inlet of the microreactor 101 is connected with the second raw material container 103 via the second connecting section 111. The second liquid delivery pump 106 delivers the second raw material from the second raw material container 103 to the second inlet of the microreactor 101.

The outlet of the microreactor 101 is connected with the product collection container 104 via the residence section 112. The product collection container 104 is a container for collecting the product fluid generated in the microreactor 101 and the subsequent residence section 112. Note that, as necessary, a fluid that is necessary for dilution, neutralization, or the like of the product fluid can be stored in the product collection container 104. In addition, as necessary, the length of the residence section 112 can be set to the minimum length for taking out the product fluid generated in the microreactor 101 into the product collection container 104, in order to minimize raw materials to be used.

For example, syringe pumps, tube pumps, plunger pumps, diaphragm pumps, screw pumps, manual liquid delivery using a syringe, liquid delivery using a water head difference, and the like can be used as for the first liquid delivery pump 105 and the second liquid delivery pump 106. Note that, in a case where a syringe pump is used as the first liquid delivery pump 105 or the second liquid delivery pump 106, a syringe in which the first raw material or the second raw material is prepared can be used as the first raw material container 102 or the second raw material container 103 in order to minimize the raw material to be used.

As materials of the microreactor 101, the first raw material container 102, the second raw material container 103, the product collection container 104, the first connecting section 110, the second connecting section 111, the residence section 112, liquid-contact sections of the pumps, syringes, diaphragms, fittings, and the like, appropriate materials can be used depending on the types of the fluids as long as those materials do not adversely affect the first raw material, the second raw material, and the product fluid, and are resistant to deterioration caused by the first raw material, the second raw material, and the product fluid.

These materials may be identical to each other or may be different from each other for each installation location in the system. The materials can be selected as appropriate depending on processability, flexibility, and the like.

Examples of the material of the microreactor 101 include stainless steel, gold, glass, Hastelloy, ceramic, PE (polyethylene), PP (polypropylene), TPX (polymethylpentene), PDMS (polydimethylsiloxane), PC (polycarbonate), fluorine resins such as PTFE (polytetrafluoroethylene) and PFA (perfluoroalkoxyalkane), and the like.

In order to enhance corrosion resistance, chemical resistance, and the like, the material of the microreactor 101 may include linings of glass or the like, coatings of nickel, gold, or the like, or oxide films formed using silicon oxidation.

As the temperature adjusting apparatuses 115, 116, 117, and 118, appropriate apparatuses such as a heat exchanger using a heat transfer medium, a thermostatic water bath using a heat transfer medium, a Peltier-type temperature control apparatus, and a mantle heater can be used. As the heat transfer media, water, ethylene glycol, a water/ethylene glycol mixed solvent, dry ice and a water/ethanol mixed solvent, dry ice and a water/methanol mixed solvent, and the like can be used.

The temperatures of the temperature adjusting apparatus 115, the temperature adjusting apparatus 116, the temperature adjusting apparatus 117, and the temperature adjusting apparatus 118 may be adjusted to fall within mutually the same temperature range, or may be adjusted to fall within mutually different temperature ranges. The temperatures of the temperature adjusting apparatuses 115, 116, 117, and 118 can be adjusted according to process rates, compound stability, and the like. Note that, in a case where processes are performed at room temperature or in some cases, the temperature adjusting apparatuses 115, 116, 117, and 118 may not be provided.

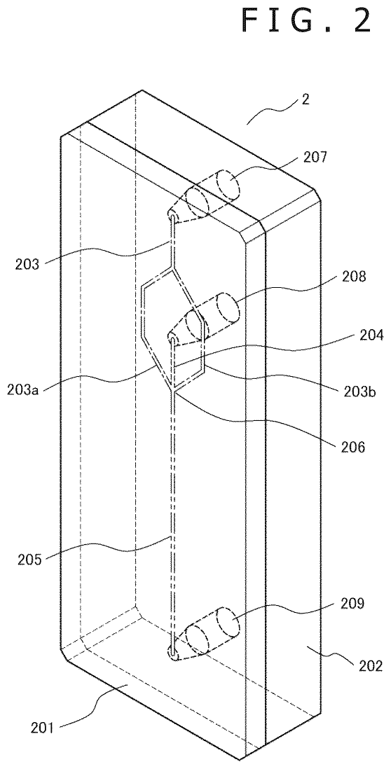

FIG. 2 is a figure depicting an example of the microreactor.

As depicted in FIG. 2, as the microreactor 101 to be used for the condition-exploration microreactor system 1, a microreactor 2 that can mix fluids at mutually different flow rates may also be used.

The microreactor 2 that can mix fluids at mutually different flow rates has: two inlets (207 and 208) from which individual fluids are introduced from the outside; micro flow paths (203, 204, and 205) where the introduced fluids are caused to merge; and a fluid outlet 209 from which a product fluid generated by the merging at a confluence 206 flows out to the outside.

The microreactor 2 includes an upper plate 201 and a lower plate 202. Grooves are machined into the upper plate 201, and the lower plate 202 is placed thereon so as to cover the grooves, thereby forming the micro flow paths (203, 204, and 205). Such grooves can be formed in either the upper plate 201 or the lower plate 202.

The lower plate 202 is provided with through-holes (207, 208, and 209) at positions that overlap the respective terminal ends of the micro flow paths (203, 204, and 205). As the through-holes (207, 208, and 209), the high-flow-rate side fluid inlet 207, the low-flow-rate side fluid inlet 208, and the fluid outlet 209 penetrate from the side of the micro flow paths (203, 204, and 205) to a surface of the lower plate 202 on the side opposite to the micro flow paths (203, 204, and 205).

Screw grooves which are not illustrated can be formed on the through-holes (207, 208, and 209). The tubes of the first connecting section 110, the second connecting section 111, and the residence section 112 can be connected via fittings that can be screwed with the screw grooves. Alternatively, as another possible configuration, the tubes may be directly connected to the through-holes (207, 208, and 209) without using fittings. In addition, as still another possible configuration, the fittings of the first connecting section 110, the second connecting section 111, and the residence section 112 are connected by screwing the fittings with the screw grooves.

The micro flow paths (203, 204, and 205) include: the high-flow-rate side flow path 203 extending from the high-flow-rate side fluid inlet 207 to reach the confluence 206; the low-flow-rate side flow path 204 extending from the low-flow-rate side fluid inlet 208 to reach the confluence 206; and the mixing flow path 205 extending from the confluence 206 to reach the fluid outlet 209.

The high-flow-rate side flow path 203 is used for delivering a fluid that is set to a high mixing ratio and a relatively high flow rate in the fluids to be mixed in the microreactor 2. On the other hand, the low-flow-rate side flow path 204 is used for delivering a fluid that is set to a low mixing ratio and a relatively low flow rate in the fluids to be mixed in the microreactor 2.

In the microreactor 2, the high-flow-rate side fluid is introduced from the high-flow-rate side fluid inlet 207, flows through the high-flow-rate side flow path 203, and arrives at the confluence 206. The low-flow-rate side fluid is introduced from the low-flow-rate side fluid inlet 208, flows through the low-flow-rate side flow path 204, and arrives at the confluence 206. The high-flow-rate side fluid and the low-flow-rate side fluid merge at the confluence 206, and initiate being mixed and reacting with each other. These fluids flow through the mixing flow path 205, and are discharged to the outside from the fluid outlet 209.

The high-flow-rate side flow path 203 is provided such that the total flow path volume is greater than the total flow path volume of the low-flow-rate side flow path 204. For example, the high-flow-rate side flow path 203 is provided to have the flow path length longer than the flow path length of the low-flow-rate side flow path 204 having an flow path width and flow path depth equivalent to those of the high-flow-rate side flow path 203. According to such a structure, in a case where the mixing ratio is biased toward the side of one fluid, and the fluids are controlled to flow at flow rates which are significantly different from each other, it is possible to reduce the mismatch between the timings at which the fluids arrive at the confluence 206.

The high-flow-rate side flow path 203 branches into two symmetric branch flow paths 203a and 203b at an intermediate section, and the symmetric branch flow paths 203a and 203b merge with each other at the confluence 206. The low-flow-rate side flow path 204 is connected to the confluence 206 between the two branch flow paths 203a and 203b. At the confluence 206, the low-flow-rate side fluid and the high-flow-rate side fluid that flow in from the upstream side flow to the mixing flow path 205 on the downstream side. According to such a structure, the low-flow-rate side fluid merges with the high-flow-rate side fluid and initiates being mixed therewith in a state where the low-flow-rate side fluid is sandwiched by the high-flow-rate side fluid. By causing the low-flow-rate side fluid to be sandwiched by the high-flow-rate side fluid, the area of interface between the fluids is expanded, and accordingly, the mixing efficiency can be enhanced.

Preferably, the high-flow-rate side flow path 203, the low-flow-rate side flow path 204, and the mixing flow path 205 are provided such that the flow path diameters, flow path widths, or flow path depths of the high-flow-rate side flow path 203, the low-flow-rate side flow path 204, and the mixing flow path 205 are equal to or smaller than 2 mm. If the high-flow-rate side flow path 203, the low-flow-rate side flow path 204, and the mixing flow path 205 are such flow paths, the effects of the micro reaction field such as enhancement of surface effects and heat transfer rates can be attained sufficiently. In particular, preferably, the high-flow-rate side flow path 203, the low-flow-rate side flow path 204, the confluence 206, and the mixing flow path 205 are provided such that the flow path diameters, flow path widths, or flow path depths of the high-flow-rate side flow path 203 and the low-flow-rate side flow path 204 immediately before the confluence 206, the confluence 206, and the mixing flow path 205 are equal to or greater than 10 μm and equal to or smaller than 1 mm. If the high-flow-rate side flow path 203, the low-flow-rate side flow path 204, the confluence 206, and the mixing flow path 205 are such flow paths, the fluids can be mixed uniformly and rapidly by molecular diffusion.

Note that the microreactor 2 that can mix the fluids at mutually different flow rates can also be used in a case where the flow rate ratio of the fluids is set to 1:1. The mixing of the fluids may take the form of uniform mixing of the fluids, or may take the form of non-uniform mixing of the fluids, for example, the form in which a plurality of phases of an emulsified state or the like are formed.

In FIG. 2, the microreactor 2 includes the high-flow-rate side flow path 203 and the low-flow-rate side flow path 204 having predetermined shapes. It should be noted that a microreactor used for the condition-exploration microreactor system 1 can be provided in an appropriate shape as long as the microreactor has micro flow paths where at least two fluids are mixed. For example, micro flow paths can be provided in a Y-shape, a T-shape, a shape forming multi-layer flows to be merged, or the like.

A microreactor used for the condition-exploration microreactor system 1 may have flow path volumes until the two fluids merge, which volumes are different from each other or equivalent to each other. Flow paths of a microreactor used for the condition-exploration microreactor system 1 do not necessarily need to be entirely micro flow paths. Flow paths of a microreactor used for the condition-exploration microreactor system 1 have flow path diameters, flow path widths, or flow path depths that can be changed depending on the types of reactions or the like.

Next, a process condition optimization method using the condition-exploration microreactor system 1 is explained.

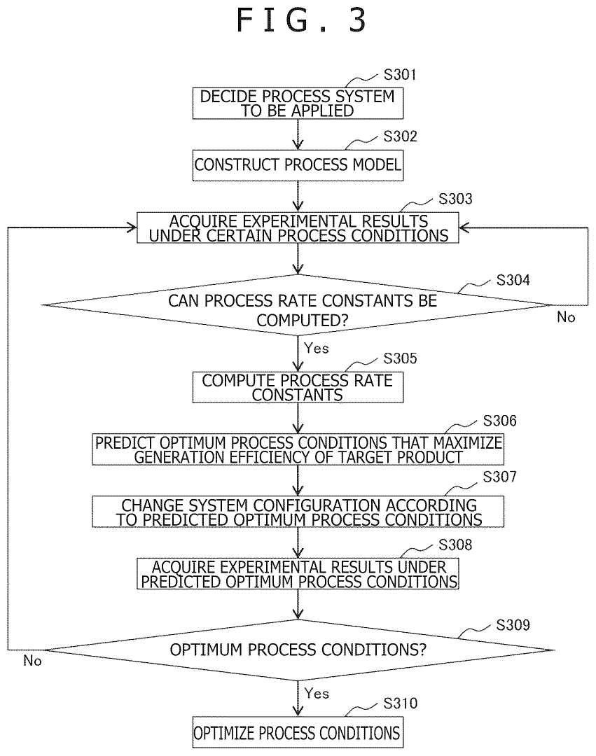

FIG. 3 is a flowchart depicting an example of optimization of process conditions.

In the condition-exploration microreactor system 1, a microreactor is used for mixing the first raw material and the second raw material. In the microreactor, the first raw material and the second raw material are introduced into micro flow paths which are micro reaction fields, and then a process in which the first raw material and the second raw material are mixed in the micro flow paths to generate a product fluid from the first raw material and the second raw material is initiated.

By using the microreactor, the flow rate ratio between the first raw material and the second raw material, the process initiation timing, and the process end timing can be controlled strictly. Accordingly, precise control of the mixing ratio between the first raw material and the second raw material and precise control of the process time are possible. Accordingly, a desired product can be generated with a high yield. In view of this, a process system which is to be applied to the condition-exploration microreactor system 1 and for which process conditions are desired to be optimized is decided (Step S301).

Next, in the condition-exploration microreactor system 1, the operation/analysis section 108 decomposes a process to be applied into elementary processes, and constructs a process model including process rate equations for the respective elementary processes (Step S302).

Here, the first raw material is prepared in the first raw material container 102, and the second raw material is prepared in the second raw material container 103.

First, according to an instruction displayed on the operation/analysis section 108, the system configuration on the latter stage side of the first liquid delivery pump 105 and the second liquid delivery pump 106 is set, and then process conditions reflecting the system configuration are input, or the system configuration on the latter stage side of the first liquid delivery pump 105 and the second liquid delivery pump 106 is set according to displayed process conditions.

The first raw material prepared in the first raw material container 102 is delivered from the first raw material container 102 to the first inlet of the microreactor 101 by the first liquid delivery pump 105. In addition, the second raw material prepared in the second raw material container 103 is delivered from the second raw material container 103 to the second inlet of the microreactor 101 by the second liquid delivery pump 106.

Next, the first raw material and the second raw material are mixed in the microreactor 101. By the mixing, a process of the first raw material and the second raw material is initiated. The process of the first raw material and the second raw material further progresses while a product fluid generated in the microreactor 101 flows in the subsequent residence section 112 toward downstream.

Next, the product fluid discharged from the microreactor 101 and the subsequent residence section 112 is collected into the product collection container 104. The process is initiated in the micro flow paths of the microreactor 101, and proceeds through the subsequent residence section 112.

An amount of the product fluid collected in the product collection container 104 that is required for performing an analysis in the analyzing section 109 is delivered to the analyzing section 109 by the sampling for analysis 114. The analyzing section 109 measures constituents of the first raw material, constituents of the second raw material, and values according to the process of the constituents of the first raw material and the constituents of the second raw material.

The data obtained at the analyzing section 109 is sent to the operation/analysis section 108 through the signal line 113, and experimental results obtained under certain process conditions are acquired (Step S303).

Here, on the basis of the constructed process model, it is determined whether process rate constants can be computed, from the number of pieces of obtained experiment data (Step S304). Since the process model consists of simultaneous differential equations, typically, the number of experimental conditions under which data is obtained is required to be at least “the number of process rate constants that are desired to be determined +1.” In a case where the number of pieces of obtained experiment data is obviously smaller than the number of process rate constants that are desired to be determined, there is a fear that the process rate constants are not determined uniquely.

In a case where the number of pieces of obtained data has not reached the number required to be able to compute the process rate constants (No), the acquisition of experimental results is further continued (Step S303). In a case where the number of pieces of obtained data has reached the number required to be able to compute the process rate constants (Yes), the process rate constants in the process model are computed by solving the inverse problem so as to reproduce the obtained data (Step S305).

Time series data of the generation efficiency of the product of the process of the first raw material and the second raw material is calculated from the determined process rate constants, and optimum process conditions that maximize the generation efficiency of the target product are predicted from the process time dependency of the product (Step S306).

Here, since the time series data of substances included in the process model is obtained, continuity of factors can be ensured within the scope of the process model, and even values of factors that have not been experimented (process time, process temperature, raw material concentrations, etc.) can be predicted. In addition, even in a case where intermediate products that cannot be measured with an analyzing apparatus are generated, or even in a case where, although intermediate products can be measured with an analyzing apparatus, the intermediate products cannot be effectively measured because the analysis speed cannot keep up due to the short lifetimes of the intermediate products, optimum process conditions can be predicted for substances if the substances are included in the process model.

The process conditions that are predicted to maximize the generation efficiency of the target product are used as the next experimental conditions, and the next experimental conditions are displayed, as a change instruction, on the operation/analysis section 108. According to the displayed change instruction, the system configuration on the latter stage side of the first liquid delivery pump 105 and the second liquid delivery pump 106 is changed (Step S307). Items of the change instruction displayed on the operation/analysis section 108 differ depending on the type of process. Examples of the items include: the flow rates of the first solution and the second solution; the temperatures of the temperature adjusting apparatuses 115, 116, 117, and 118; the internal diameter and length of the residence section 112; the types of the first raw material and the second raw material; the concentrations (constituent ratios) of the first raw material and the second raw material; the solvents of the first raw material and the second raw material; and the like, and some of these.

According to the process conditions of the predicted optimum process, the first raw material prepared in the first raw material container 102 is delivered from the first raw material container 102 to the first inlet of the microreactor 101 by the first liquid delivery pump 105. In addition, the second raw material prepared in the second raw material container 103 is delivered from the second raw material container 103 to the second inlet of the microreactor 101 by the second liquid delivery pump 106.

Next, the first raw material and the second raw material are mixed in the microreactor 101. By the mixing, a process of the first raw material and the second raw material is initiated. The process of the first raw material and the second raw material further progresses while a product fluid generated in the microreactor 101 flows in the subsequent residence section 112 toward downstream.

Next, the product fluid discharged from the microreactor 101 and the subsequent residence section 112 is collected into the product collection container 104. The process is initiated in the micro flow paths of the microreactor 101, and proceeds through the subsequent residence section 112.

An amount of the product fluid collected in the product collection container 104 that is required for performing an analysis in the analyzing section 109 is delivered to the analyzing section 109 by the sampling for analysis 114. The analyzing section 109 measures the constituents of the first raw material, the constituents of the second raw material, and the values according to a process of the constituents of the first raw material and the constituents of the second raw material.

The data obtained at the analyzing section 109 is sent to the operation/analysis section 108 through the signal line 113, and experimental results obtained under the predicted optimum process conditions are acquired (Step S308).

Here, it is determined whether the obtained experimental results fall within the desired range of the generation efficiency of the target product, and it is determined whether the conditions are the predicted optimum process conditions (Step S309).

In a case where the obtained experimental results do not fall within the desired range of the generation efficiency of the target product (No), the acquisition of experimental results is further continued (Step S303). In a case where the obtained experimental results fall within the desired range of the generation efficiency of the target product (Yes), it is determined that the process conditions have been optimized (Step S310).

The following explains the process condition optimization method using the condition-exploration microreactor system 1, with use of specific examples.

### Specific Example 1

### Condition Optimization of Lipid Nanoparticle (Lipid NanoParticle: LNP) Generation Process

The LNP generation process is a process that generates LNPs in which an ionizable lipid, a PEG (Polyethylene glycol: polyethylene glycol) lipid, a phospholipid, and cholesterol encapsulate reverse micelles containing cholesterol and an ionizable lipid confining an aqueous solution containing a nucleic acid (RNA (Ribonucleic Acid: ribonucleic acid), DNA (Deoxyribonucleic Acid: deoxyribonucleic acid) ), a low molecule compound, and the like by preparing the aqueous solution containing the nucleic acid and the like, and an ethanol solution containing the ionizable lipid, the PEG lipid, the phospholipid, and the cholesterol, for example, and rapidly mixing them.

In the condition-exploration microreactor system 1, a microreactor is used for mixing the aqueous solution and the ethanol solution. In the microreactor, the aqueous solution and the ethanol solution are introduced into the micro flow paths which are micro reaction fields, and the LNP generation process is initiated by mixing them in the micro flow paths.

By using the microreactor, the flow rate ratio between the aqueous solution and the ethanol solution, the LNP generation process initiation timing, and the LNP generation process end timing can be controlled strictly. Accordingly, precise control of the mixing ratio between the nucleic acid and the like which are the constituents of the aqueous solution and the ionizable lipid, the PEG lipid, the phospholipid, and the cholesterol which are the constituents of the ethanol solution, and precise control of the process time are possible. Accordingly, LNP having the desired particle size and a high encapsulation ratio of the nucleic acid and the like can be generated with a high yield.

In the condition-exploration microreactor system 1, the operation/analysis section 108 constructs an LNP generation process model including process rate equations including process rate constants for the reverse micellization process and process rate equations including process rate constants for the LNP encapsulation process.

The aqueous solution containing the nucleic acid and the like is prepared in the first raw material container 102. The ethanol solution containing the ionizable lipid, the PEG lipid, the phospholipid, and the cholesterol is prepared in the second raw material container 103.

First, the aqueous solution prepared in the first raw material container 102 is delivered from the first raw material container 102 to the first inlet of the microreactor 101 by the first liquid delivery pump 105. In addition, the ethanol solution prepared in the second raw material container 103 is delivered from the second raw material container 103 to the second inlet of the microreactor 101 by the second liquid delivery pump 106.

Next, the aqueous solution and the ethanol solution are mixed in the microreactor 101. By the mixing, the LNP generation process of the aqueous solution and the ethanol solution is initiated. First, the reverse micellization process in which the reverse micelle containing the ionizable lipid and the cholesterol confines the aqueous solution containing the nucleic acid and the like is initiated.

Next, the LNP encapsulation process in which the ionizable lipid, the PEG reagent, the phospholipid, and the cholesterol encapsulate the reverse micelle is initiated. The reverse micellization process and the LNP encapsulation process further progress while a product fluid generated in the microreactor 101 flows in the subsequent residence section 112 toward downstream.

Next, the product fluid discharged from the microreactor 101 and the subsequent residence section 112 is collected into the product collection container 104. The process is initiated in the micro flow paths of the microreactor 101, and proceeds through the subsequent residence section 112, and then a product fluid which is a solution containing LNP is obtained.

An amount of the product fluid collected in the product collection container 104 that is required for performing an analysis in the analyzing section 109 is delivered to the analyzing section 109 by the sampling for analysis 114. The analyzing section 109 measures the constituents of the aqueous solution, the constituents of the ethanol solution, and the values according to the process of the constituents of the aqueous solution and the constituents of the ethanol solution, such as the average particle size of LNP, the variation in LNP particle sizes, and the encapsulation ratio of the nucleic acid and the like in LNP.

The data obtained at the analyzing section 109 is sent to the operation/analysis section 108 through the signal line 113. By solving the inverse problem so as to reproduce the obtained data, the process rate constants for the reverse micellization process and the process rate constants for the LNP generation process in the LNP generation process model are computed. Time series data of the generation efficiency of the reverse micelle and LNP is calculated from the determined process rate constants, and optimum process conditions that maximize the generation efficiency of target LNP are predicted.

The process conditions that are predicted to maximize the generation efficiency of target LNP are used as the next experimental conditions, and the next experimental conditions are displayed, as a change instruction, on the operation/analysis section 108. According to the displayed change instruction, the system configuration on the latter stage side of the first liquid delivery pump 105 and the second liquid delivery pump 106 is changed. Examples of items of the change instruction displayed on the operation/analysis section 108 include: the flow rates of the aqueous solution (first solution) and the ethanol solution (second solution); the temperatures of the temperature adjusting apparatuses 115, 116, 117, and 118; the internal diameter and length of the residence section 112; the concentration (composition ratio) of each lipid; the concentrations of the nucleic acid and the like; the solvent of the lipid solution; and the like, and some of these.

### Specific Example 2

### Condition Optimization of Chemical Modification Process

The chemical modification process is a chemical reaction process performed in order to add functions of particular molecules by further adding the molecules to functional substances which are particles or molecules having particular functions. For example, there are a known bioconjugation reaction process that binds a small molecule drug with a bio-related substance such as an antibody or a peptide that recognizes a target, via linker molecules, and generates an antibody drug conjugate (ADC), a peptide drug conjugate (PDC), or the like, and a known PEGylation reaction process that adds PEG to functional substances such as bio-related substances, e.g., lipid nanoparticles such as LNP, peptides, and nucleic acid molecules.

The PEGylation reaction process is a process that generates PEGylated substances in which PEG is added to functional substances by preparing and mixing a solution containing the functional substances and a solution containing a PEGylation reagent. Here, since multiple types of PEGylated functional substances can be generated as products in a case where there are a plurality of sites on the functional substances where the PEGylation reagent can act, not all of them are necessarily target PEGylated functional substances, some of them are target products, and the rest can be by-products.

In the condition-exploration microreactor system 1, a microreactor is used for mixing the solution containing the functional substances and the solution containing the PEGylation reagent. In the microreactor, the solution containing the functional substances and the solution containing the PEGylation reagent are introduced into the micro flow paths which are micro reaction fields, and the PEGylation reaction process is initiated by mixing them in the micro flow paths.

By using the microreactor, the flow rate ratio between the solution containing the functional substances and the solution containing the PEGylation reagent, the PEGylation reaction process initiation timing, and the PEGylation reaction process end timing can be controlled strictly. Accordingly, precise control of the mixing ratio between the functional substances which are constituents of the functional substance solution and the PEGylation reagent which is a constituent of the PEGylation reagent solution, and precise control of the process time are possible. Accordingly, it is possible to generate a substance with a desired number of PEG added at desired positions of the functional substances, with a high yield.

In the condition-exploration microreactor system 1, the operation/analysis section 108 constructs a chemical modification process model including process rate equations including process rate constants for reactions that generate the target PEGylated substances and process rate equations including process rate constants for side reactions.

The solution containing the functional substances is prepared in the first raw material container 102. The solution containing the PEGylation reagent is prepared in the second raw material container 103.

First, the solution containing the PEGylation reagent prepared in the first raw material container 102 is delivered from the first raw material container 102 to the first inlet of the microreactor 101 by the first liquid delivery pump 105. In addition, the solution containing the functional substances prepared in the second raw material container 103 is delivered from the second raw material container 103 to the second inlet of the microreactor 101 by the second liquid delivery pump 106.

Next, the solution containing the PEGylation reagent and the solution containing the functional substances are mixed in the microreactor 101. By the mixing, the PEGylation reaction process of the solution containing the PEGylation reagent and the solution containing the functional substances is initiated. The PEGylation reaction process further progresses while a product fluid generated in the microreactor 101 flows in the subsequent residence section 112 toward downstream.

Next, the product fluid discharged from the microreactor 101 and the subsequent residence section 112 is collected into the product collection container 104. The process is initiated in the micro flow paths of the microreactor 101, and proceeds through the subsequent residence section 112, and then a product fluid which is a solution containing the PEGylated functional substances is obtained.

An amount of the product fluid collected in the product collection container 104 that is required for performing an analysis in the analyzing section 109 is delivered to the analyzing section 109 by the sampling for analysis 114. The analyzing section 109 measures the constituents of the solution containing the functional substances, the constituents of the solution containing the PEGylated substances, and the values according to the process of the constituents of the solution containing the functional substances and the constituents of the solution containing the PEGylated substances, such as the ratio of functional substances that have not been PEGylated and the generation ratio of a plurality of PEGylated functional substances.

The data obtained at the analyzing section 109 is sent to the operation/analysis section 108 through the signal line 113. By solving the inverse problem so as to reproduce the obtained data, process rate equations including the process rate constants for PEGylation reactions that generate the target products and process rate equations including the process rate constants for PEGylation reactions that generate by-products are computed. Time series data of the generation efficiency of the PEGylated functional substances is calculated from the determined process rate constants, and optimum process conditions that maximize the generation efficiency of the target PEGylated functional substances are predicted.

The process conditions that are predicted to maximize the generation efficiency of the target PEGylated functional substances are used as the next experimental conditions, and the next experimental conditions are displayed, as a change instruction, on the operation/analysis section 108. According to the displayed change instruction, the system configuration on the latter stage side of the first liquid delivery pump 105 and the second liquid delivery pump 106 is changed. Examples of items of the change instruction displayed on the operation/analysis section 108 include: the flow rates of the solution containing the functional substances (first solution) and the solution containing the PEGylation reagent (second solution); the temperatures of the temperature adjusting apparatuses 115, 116, 117, and 118; the internal diameter and length of the residence section 112; the concentrations of the functional substances and the PEGylation reagent; the solvents of the solution containing the functional substances and the solution containing the PEGylation reagent; and the like, and some of these.

### Specific Example 3

### Condition Optimization of Extension Reaction Process

The extension reaction process is a chemical reaction process performed in order to extend molecule chains by sequentially adding molecules. For example, there are a known peptide-chain extension reaction process that sequentially adds an amino acid by dehydration condensation, and a known nucleic-acid-chain extension reaction process that sequentially adds DNA or RNA by condensation such as dehydration condensation.

In the extension reaction process, a solution containing a recipient substance and a solution containing an additive substance are prepared. By mixing the solutions, the additive substance condenses onto the recipient substance. Here, since a plurality of substances can be generated as products in a case where there are a plurality of sites on the recipient substance where the additive substance can act, not all of them are necessarily target substances, some of them are target products, and some of them can be by-products.

In the condition-exploration microreactor system 1, a microreactor is used for mixing the solution containing the recipient substance and the solution containing the additive substance. In the microreactor, the solution containing the recipient substance and the solution containing the additive substance are introduced into the micro flow paths which are micro reaction fields, and the extension reaction process is initiated by mixing them in the micro flow paths.

By using the microreactor, the flow rate ratio between the solution containing the recipient substance and the solution containing the additive substance, the extension reaction process initiation timing, and the extension reaction process end timing can be controlled strictly. Accordingly, precise control of the mixing ratio between the recipient substance which is a constituent of the solution containing the recipient substance and the additive substance which is a constituent of the solution containing the additive substance, and precise control of the process time are possible. Accordingly, it is possible to generate a substance with the additive substance added at desired positions of the recipient substance, with a high yield.

In the condition-exploration microreactor system 1, the operation/analysis section 108 constructs an extension reaction process model including process rate equations including process rate constants for reactions that generate the target substances and process rate equations including process rate constants for side reactions.

The solution containing the recipient substance is prepared in the first raw material container 102. The solution containing the additive substance is prepared in the second raw material container 103.

First, the solution containing the recipient substance prepared in the first raw material container 102 is delivered from the first raw material container 102 to the first inlet of the microreactor 101 by the first liquid delivery pump 105. In addition, the solution containing the additive substance prepared in the second raw material container 103 is delivered from the second raw material container 103 to the second inlet of the microreactor 101 by the second liquid delivery pump 106.

Next, the solution containing the recipient substance and the solution containing the additive substance are mixed in the microreactor 101. By the mixing, the extension reaction process of the solution containing the recipient substance and the solution containing the additive substance is initiated. The extension reaction process further progresses while a product fluid generated in the microreactor 101 flows in the subsequent residence section 112 toward downstream.

Next, the product fluid discharged from the microreactor 101 and the subsequent residence section 112 is collected into the product collection container 104. The process is initiated in the micro flow paths of the microreactor 101, and proceeds through the subsequent residence section 112, and then a product fluid which is a solution containing the substance to which the additive substance has been added is obtained.

An amount of the product fluid collected in the product collection container 104 that is required for performing an analysis in the analyzing section 109 is delivered to the analyzing section 109 by the sampling for analysis 114. The analyzing section 109 measures the constituents of the solution containing the recipient substance, the constituents of the solution containing the additive substance, and the values according to the process of the constituents of the solution containing the recipient substance and the constituents of the solution containing the additive substance, such as the ratio of substances that have not been added and the generation ratios of a plurality of added substances.

The data obtained at the analyzing section 109 is sent to the operation/analysis section 108 through the signal line 113. By solving the inverse problem so as to reproduce the obtained data, process rate equations including the process rate constants for reaction processes that generate the target products and process rate equations including the process rate constants for side reaction processes are computed. Time series data of the generation efficiency of the added substance is calculated from the determined process rate constants, and optimum process conditions that maximize the generation efficiency of the target added substance are predicted.

The process conditions that are predicted to maximize the generation efficiency of the target added substance are used as the next experimental conditions, and the next experimental conditions are displayed, as a change instruction, on the operation/analysis section 108. According to the displayed change instruction, the system configuration on the latter stage side of the first liquid delivery pump 105 and the second liquid delivery pump 106 is changed. Examples of items of the change instruction displayed on the operation/analysis section 108 include: the flow rates of the solution containing the recipient substance (first solution) and the solution containing the additive substance (second solution); the temperatures of the temperature adjusting apparatuses 115, 116, 117, and 118; the internal diameter and length of the residence section 112; the concentrations of the recipient substance and the additive substance; the solvents of the solution containing the recipient substance and the solution containing the additive substance; and the like, and some of these.

The condition-exploration microreactor system 1 and the process condition optimization method using the same mentioned above make it possible to reduce the number of experiments required for the optimization of process conditions such as reactions as much as possible, to predict values for factors that have not been experimented or factors that cannot be measured, and to reduce the time required for experiments and the amount of reagents (waste) required as much as possible.

### Second Embodiment

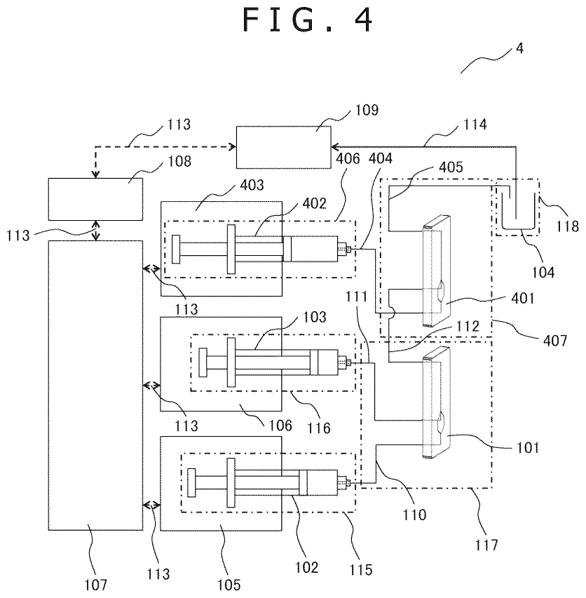

A condition-exploration microreactor system and a process condition optimization method according to a second embodiment of the present invention are explained with reference to FIG. 4.

FIG. 4 is a schematic diagram of a condition-exploration microreactor system 4 according to the present embodiment.

As depicted in FIG. 4, the condition-exploration microreactor system 4 according to the present embodiment includes, as main constituent elements, the microreactor 101, a microreactor 401, the first raw material container 102, the second raw material container 103, a third raw material container 402, the product collection container 104, the first liquid delivery pump 105, the second liquid delivery pump 106, a third liquid delivery pump 403, the control section 107, the operation/analysis section 108, the analyzing section 109, the first connecting section 110, the second connecting section 111, a third connecting section 404, the residence section 112, a residence section 405, the signal lines 113, the sampling for analysis 114, the temperature adjusting apparatus 115, the temperature adjusting apparatus 116, the temperature adjusting apparatus 117, the temperature adjusting apparatus 118, a temperature adjusting apparatus 406, and a temperature adjusting apparatus 407.

In addition, although not illustrated, the residence section 112: may include a tube, a fitting connecting the tube and the microreactor 101, and a fitting connecting the tube and the microreactor 401; may include a device in which a residence flow path is formed, a fitting connecting the device and the microreactor 101, and a fitting connecting the device and the microreactor 401; or may include a fitting directly connecting the microreactor 101 and the microreactor 401, and the like. The residence section 405: may include a tube and a fitting connecting the tube and the microreactor 401; may include a device in which a residence flow path is formed and a fitting connecting the device and the microreactor 401; may include a tube, a fitting connecting the tube and the microreactor 401, and a fitting connecting the tube and the product collection container 104; may include a device in which a residence flow path is formed, a fitting connecting the device and the microreactor 401, and a fitting connecting the device and the product collection container 104; or may include a fitting directly connecting the product collection container 104 and the microreactor 401, and the like.

Note that, in a case where functions of the residence sections 112 and 405 are not required, e.g., in a case where the processes in the microreactors 101 and 401 end instantaneously, the microreactor 101 and the microreactor 401 are directly connected without using a fitting or the like, and furthermore, the product is collected directly from the microreactor 401 into the product collection container 104. In particular, in a case where the process in the microreactor 101 or the microreactor 401 is particle generation, an excessively long residence section increases the risk of blockage in the residence section, and accordingly, a configuration in which the residence sections 112 and 405 are not provided, the microreactor 101 and the microreactor 401 are directly connected, and the microreactor 401 and the product collection container 104 are directly connected is preferable.

The first connecting section 110: may include a tube, a fitting connecting the tube and the first raw material container 102, and a fitting connecting the tube and the microreactor 101; or may include a fitting directly connecting the first raw material container 102 and the microreactor 101, and the like. In addition, the second connecting section 111: may include a tube, a fitting connecting the tube and the second raw material container 103, and a fitting connecting the tube and the microreactor 101; or may include a fitting directly connecting the second raw material container 103 and the microreactor 101, and the like. Furthermore, the third connecting section 404: may include a tube, a fitting connecting the tube and the third raw material container 402, and a fitting connecting the tube and the microreactor 401; or may include a fitting directly connecting the third raw material container 402 and the microreactor 401, and the like.

The temperature adjusting apparatus 115, the temperature adjusting apparatus 116, the temperature adjusting apparatus 117, the temperature adjusting apparatus 118, the temperature adjusting apparatus 406, and the temperature adjusting apparatus 407 are provided to adjust predetermined areas in the system to predetermined temperatures, as represented by broken lines in the figure. The first raw material container 102 is included in the adjustment range of the temperature adjusting apparatus 115. The second raw material container 103 is included in the adjustment range of the temperature adjusting apparatus 116. The third raw material container 402 is included in the adjustment range of the temperature adjusting apparatus 406. Some of the first connecting section 110, the second connecting section 111, the microreactor 101, and the residence section 112 are included in the adjustment range of the temperature adjusting apparatus 117. Some of the third connecting section 404, the microreactor 401, the residence section 405, and the residence section 112 are included in the adjustment range of the temperature adjusting apparatus 407. The product collection container 104 is included in the adjustment range of the temperature adjusting apparatus 118.

The microreactor 101 and the microreactor 401 are flow-type reactors and have two inlets from which individual fluids are introduced from the outside, micro flow paths where the introduced fluids are caused to merge, and an outlet from which a product fluid generated by the merging flows out to the outside. The microreactor 101 and the microreactor 401 mix, in the micro flow paths, the fluid introduced from the first inlet and the fluid introduced from the second inlet. By mixing the fluids, a product fluid that has initiated a predetermined process is generated.

A first raw material is prepared in the first raw material container 102. The first inlet of the microreactor 101 is connected with the first raw material container 102 via the first connecting section 110. The first liquid delivery pump 105 delivers the first raw material from the first raw material container 102 to the first inlet of the microreactor 101.

A second raw material is prepared in the second raw material container 103. The second inlet of the microreactor 101 is connected with the second raw material container 103 via the second connecting section 111. The second liquid delivery pump 106 delivers the second raw material from the second raw material container 103 to the second inlet of the microreactor 101.

A third raw material is prepared in the third raw material container 402. The second inlet of the microreactor 401 is connected with the third raw material container 402 via the third connecting section 404. The third liquid delivery pump 403 delivers the third raw material from the third raw material container 402 to the second inlet of the microreactor 401.

The outlet of the microreactor 101 is connected with the first inlet of the microreactor 401 via the residence section 112.

The outlet of the microreactor 401 is connected with the product collection container 104 via the residence section 405. The product collection container 104 is a container for collecting the product fluid generated in the microreactor 101, the subsequent residence section 112, the microreactor 401, and the subsequent residence section 405. Note that, as necessary, a fluid that is necessary for dilution, neutralization, or the like of the product fluid can be stored in the product collection container 104. In addition, as necessary, the length of the residence section 405 can be set to the minimum length for taking out the product fluid generated in the microreactor 401 into the product collection container 104, in order to minimize raw materials to be used.

For example, syringe pumps, tube pumps, plunger pumps, diaphragm pumps, screw pumps, manual liquid delivery using a syringe, liquid delivery using a water head difference, and the like can be used as or for the first liquid delivery pump 105, the second liquid delivery pump 106, and the third liquid delivery pump 403. Note that, in a case where a syringe pump is used as the first liquid delivery pump 105, the second liquid delivery pump 106, or the third liquid delivery pump 403, a syringe in which the first raw material, the second raw material, or the third raw material is prepared can be used as the first raw material container 102, the second raw material container 103, or the third raw material container 402 in order to minimize the raw material to be used.

As materials of the microreactor 101, the microreactor 401, the first raw material container 102, the second raw material container 103, the third raw material container 402, the product collection container 104, the first connecting section 110, the second connecting section 111, the third connecting section 404, the residence section 112, the residence section 405, liquid-contact sections of the pumps, syringes, diaphragms, fittings, and the like, appropriate materials can be used depending on the types of the fluids as long as those materials do not adversely affect the first raw material, the second raw material, the third raw material, and the product fluid, and are resistant to deterioration caused by the first raw material, the second raw material, the third raw material, and the product fluid.

These materials may be identical to each other or may be different from each other for each installation location in the system. The materials can be selected as appropriate depending on processability, flexibility, and the like.

Examples of the materials of the microreactor 101 and the microreactor 401 include stainless steel, gold, glass, Hastelloy, ceramic, PE (polyethylene), PP (polypropylene), TPX (polymethylpentene), PDMS (polydimethylsiloxane), PC (polycarbonate), fluorine resins such as PTFE (polytetrafluoroethylene) and PFA (perfluoroalkoxyalkane), and the like.

In order to enhance corrosion resistance, chemical resistance, and the like, the materials of the microreactor 101 and the microreactor 401 may include linings of glass or the like, coatings of nickel, gold, or the like, or oxide films formed using silicon oxidation.

As the temperature adjusting apparatuses 115, 116, 117, 118, 406, and 407, appropriate apparatuses such as a heat exchanger using a heat transfer medium, a thermostatic water bath using a heat transfer medium, a Peltier-type temperature control apparatus, and a mantle heater can be used. As the heat transfer media, water, ethylene glycol, a water/ethylene glycol mixed solvent, dry ice and a water/ethanol mixed solvent, dry ice and a water/methanol mixed solvent, and the like can be used.

The temperatures of the temperature adjusting apparatus 115, the temperature adjusting apparatus 116, the temperature adjusting apparatus 117, the temperature adjusting apparatus 118, the temperature adjusting apparatus 406, and the temperature adjusting apparatus 407 may be adjusted to fall within mutually the same temperature range, or may be adjusted to fall within mutually different temperature ranges. The temperatures of the temperature adjusting apparatuses 115, 116, 117, 118, 406, and 407 can be adjusted according to process rates, compound stability, and the like. Note that, in a case where processes are performed at room temperature or in some cases, the temperature adjusting apparatuses 115, 116, 117, 118, 406, and 407 may not be included.

As the microreactor 101 and the microreactor 401 to be used for the condition-exploration microreactor system 4, the microreactor 2 that can mix fluids at mutually different flow rates can be used also. It should be noted that a microreactor used for the condition-exploration microreactor system 4 can be provided in an appropriate shape as long as the microreactor has micro flow paths at least where two fluids are mixed. For example, micro flow paths can be provided in a Y-shape, a T-shape, a shape forming multi-layer flows to be merged, or the like.

A microreactor used for the condition-exploration microreactor system 4 may have flow path volumes until the two fluids merge that are different from each other or equivalent to each other. Flow paths of a microreactor used for the condition-exploration microreactor system 4 do not necessarily need to be entirely micro flow paths. Flow paths of a microreactor used for the condition-exploration microreactor system 4 have flow path diameters, flow path widths, or flow path depths that can be changed depending on the types of reactions or the like.

The following explains the process condition optimization method using the condition-exploration microreactor system 4 using specific examples.

### Specific Example 4

### Condition Optimization of Lipid Nanoparticle (Lipid NanoParticle: LNP) Generation Process

In the condition-exploration microreactor system 4, a microreactor is used for mixing an aqueous solution containing a nucleic acid and the like, and, for example, an ethanol solution containing an ionizable lipid, a PEG lipid, a phospholipid, and cholesterol. Furthermore, a microreactor is used also for mixing the obtained fluid and an aqueous solution not containing the nucleic acid and the like.

In the first microreactor, the aqueous solution containing the nucleic acid and the like and the ethanol solution are introduced into the micro flow paths which are micro reaction fields, and the LNP generation process is initiated by mixing them in the micro flow paths. Furthermore, in the second microreactor, the obtained fluid and the aqueous solution not containing the nucleic acid and the like are introduced into the micro flow paths which are micro reaction fields, and the LNP generation process is progressed by mixing them in the micro flow paths.

By using the microreactors, the flow rate ratio between the aqueous solution containing the nucleic acid and the like and the ethanol solution, the flow rate ratio between the fluid obtained in the first microreactor and the aqueous solution not containing the nucleic acid and the like, the LNP generation process initiation timing, and the LNP generation process end timing can be controlled strictly. Accordingly, precise control of the mixing ratio between the nucleic acid and the like which are the constituents of the aqueous solution containing the nucleic acid and the like and the ionizable lipid, the PEG lipid, the phospholipid, and the cholesterol which are the constituents of the ethanol solution, precise control of the mixing ratio between water which is a constituent of the aqueous solution not containing the nucleic acid and the like and ethanol which is a constituent of the ethanol solution, and precise control of the process time are possible. Accordingly, LNP having the desired particle size and a high encapsulation ratio of the nucleic acid and the like can be generated with a high yield.

In the condition-exploration microreactor system 4, the operation/analysis section 108 constructs an LNP generation process model including process rate equations including process rate constants for the reverse micellization process and process rate equations including process rate constants for the LNP encapsulation process.

The aqueous solution containing the nucleic acid and the like is prepared in the first raw material container 102. The ethanol solution containing the ionizable lipid, the PEG lipid, the phospholipid, and the cholesterol is prepared in the second raw material container 103. The aqueous solution not containing the nucleic acid and the like is prepared in the third raw material container 402.

First, the aqueous solution containing the nucleic acid and the like prepared in the first raw material container 102 is delivered from the first raw material container 102 to the first inlet of the microreactor 101 by the first liquid delivery pump 105. In addition, the ethanol solution prepared in the second raw material container 103 is delivered from the second raw material container 103 to the second inlet of the microreactor 101 by the second liquid delivery pump 106. Furthermore, the aqueous solution not containing the nucleic acid and the like prepared in the third raw material container 402 is delivered from the third raw material container 402 to the first inlet of the microreactor 401 by the third liquid delivery pump 403.

Next, the aqueous solution containing the nucleic acid and the like and the ethanol solution are mixed in the microreactor 101. By the mixing, the lipid nanoparticle generation process of the aqueous solution containing the nucleic acid and the like and the ethanol solution is initiated. First, the reverse micellization process in which the reverse micelle containing the ionizable lipid and the cholesterol confines the aqueous solution containing the nucleic acid and the like is initiated.

Next, the LNP encapsulation process in which the ionizable lipid, the PEG reagent, the phospholipid, and the cholesterol encapsulate the reverse micelle is initiated. The reverse micellization process and the LNP encapsulation process further progress while a product fluid generated in the microreactor 101 flows in the subsequent residence section 112 toward downstream.

Next, the aqueous solution not containing the nucleic acid and the like and the fluid obtained by the mixing in the microreactor 101 are mixed in the microreactor 401. By the mixing, the LNP encapsulation process is accelerated. The LNP encapsulation process further progress while a product fluid generated in the microreactor 401 flows in the subsequent residence section 405 toward downstream.

Next, the product fluid discharged from the microreactor 401 and the subsequent residence section 405 is collected into the product collection container 104. The process is initiated in the micro flow paths of the microreactor 101, proceeds through the subsequent residence section 112, proceeds through the micro flow paths of the microreactor 401, and proceeds through the subsequent residence section 405, and then a product fluid which is a solution containing LNP is obtained.

An amount of the product fluid collected in the product collection container 104 that is required for performing an analysis in the analyzing section 109 is delivered to the analyzing section 109 by the sampling for analysis 114. The analyzing section 109 measures the constituents of the aqueous solution containing the nucleic acid and the like, the constituents of the ethanol solution, the constituents of the aqueous solution not containing the nucleic acid and the like, the values according to the process of the constituents of the aqueous solution containing the nucleic acid and the like and the constituents of the ethanol solution, and the values according to the process of the constituents of the aqueous solution not containing the nucleic acid and the like and the constituents of the fluid obtained by the mixing in the microreactor 101, such as the average particle size of LNP, the variation in LNP particle sizes, and the encapsulation ratio of the nucleic acid and the like in LNP.

The data obtained at the analyzing section 109 is sent to the operation/analysis section 108 through the signal line 113. By solving the inverse problem so as to reproduce the obtained data, the process rate constants for the reverse micellization process and the process rate constants for the LNP generation process in the LNP generation process model are computed. Time series data of the generation efficiency of the reverse micelle and LNP is calculated from the determined process rate constants, and optimum process conditions that maximize the generation efficiency of target LNP are predicted.

The process conditions that are predicted to maximize the generation efficiency of target LNP are used as the next experimental conditions, and the next experimental conditions are displayed, as a change instruction, on the operation/analysis section 108. According to the displayed change instruction, the system configuration on the latter stage side of the first liquid delivery pump 105, the second liquid delivery pump 106, and the third liquid delivery pump 403 is changed. Examples of items of the change instruction displayed on the operation/analysis section 108 include: the flow rates of the aqueous solution containing the nucleic acid and the like (first solution), the ethanol solution (second solution), and the aqueous solution not containing the nucleic acid and the like (third solution); the temperatures of the temperature adjusting apparatuses 115, 116, 117, 118, 406, and 407; the internal diameter and length of the residence section 112; the internal diameter and length of the residence section 405; the concentration (composition ratio) of each lipid; the concentrations of the nucleic acid and the like; the solvent of the lipid solution; and the like, and some of these.

### Specific Example 5

### Condition Optimization of Chemical Modification Process

The bioconjugation reaction process, which is one of chemical modification processes, is a process in which a solution containing a bio-related substance such as an antibody or a peptide, a solution containing linker molecules, and a solution containing a small molecule drug are prepared and mixed, thereby generating a drug conjugate in which the small molecule drug is bound to the bio-related substance via the linker molecules. Here, since multiple types of drug conjugates mediated by the linker molecules can be generated as products in a case where there are a plurality of sites on the bio-related substance where the linker molecules can act, not all of them are necessarily target drug conjugates, some of them are target products, and the rest can be by-products.

In the condition-exploration microreactor system 4, a microreactor is used for mixing the solution containing the bio-related substance and the solution containing the linker molecules. Furthermore, a microreactor is used also for mixing the obtained fluid and the solution containing the small molecule drug.

In the first microreactor, the solution containing the bio-related substance and the solution containing the linker molecules are introduced into the micro flow paths which are micro reaction fields, and the linker molecule binding reaction process is initiated by mixing them in the micro flow paths. Furthermore, in the second microreactor, the obtained fluid and the solution containing the small molecule drug are introduced into the micro flow paths which are micro reaction fields, and the small molecule drug binding reaction process is progressed by mixing them in the micro flow paths.

By using the microreactors, the flow rate ratio between the solution containing the bio-related substance and the solution containing the linker molecules, the flow rate ratio between the solution containing the small molecule drug and the obtained fluid, the linker molecule binding reaction process time, and the small molecule drug binding reaction process time can be controlled strictly. Accordingly, precise control of the mixing ratio between the bio-related substance and the linker molecules, precise control of the mixing ratio between the substance obtained in the first microreactor and the small molecule drug, precise control of the linker molecule binding reaction process time, and precise control of the small molecule drug binding reaction process time are possible. Accordingly, the drug conjugate in which the small molecule drug is bound at desired positions of the bio-related substance via the linker molecules can be generated with a high yield.

In the condition-exploration microreactor system 4, the operation/analysis section 108 constructs a chemical modification process model including process rate equations including process rate constants for the linker molecule binding reaction process and process rate equations including process rate constants for the small molecule drug binding reaction process.

The solution containing the bio-related substance is prepared in the first raw material container 102. The solution containing the linker molecules is prepared in the second raw material container 103. The solution containing the small molecule drug is prepared in the third raw material container 402.

First, the solution containing the bio-related substances prepared in the first raw material container 102 is delivered from the first raw material container 102 to the first inlet of the microreactor 101 by the first liquid delivery pump 105. In addition, the solution containing the linker molecules prepared in the second raw material container 103 is delivered from the second raw material container 103 to the tsecond inlet of the microreactor 101 by the second pump 106. Furthermore, the solution containing the small molecule drug prepared in the third raw material container 402 is delivered from the third raw material container 402 to the first inlet of the microreactor 401 by the third liquid delivery pump 403.

Next, the solution containing the bio-related substance and the solution containing the linker molecules are mixed in the microreactor 101. By the mixing, the linker molecule binding reaction process of the solution containing the bio-related substance and the solution containing the linker molecules is initiated. The linker molecule binding reaction process further progresses while a product fluid generated in the microreactor 101 flows in the subsequent residence section 112 toward downstream.

Next, the solution containing the small molecule drug and the fluid obtained by the mixing in the microreactor 101 are mixed in the microreactor 401. By the mixing, the small molecule drug binding reaction process of the solution containing the small molecule drug and the fluid obtained by the mixing in the microreactor 101 is initiated. The small molecule drug binding reaction process further progresses while the product fluid generated in the microreactor 101 flows in the subsequent residence section 405 toward downstream.

Next, the product fluid discharged from the microreactor 401 and the subsequent residence section 405 is collected into the product collection container 104. The process is initiated in the micro flow paths of the microreactor 101, proceeds through the subsequent residence section 112, proceeds through the micro flow paths of the microreactor 401, and proceeds through the subsequent residence section 405, and then a product fluid which is a solution containing a drug conjugate in which the small molecule drug is bound to the bio-related substance via the linker molecules is obtained.

An amount of the product fluid collected in the product collection container 104 that is required for performing an analysis in the analyzing section 109 is delivered to the analyzing section 109 by the sampling for analysis 114. The analyzing section 109 measures the constituents of the solution containing the bio-related substance, the constituents of the solution containing the linker molecules, the constituents of the solution containing the small molecule drug, the values according to the process of the constituents of the solution containing the bio-related substance and the constituents of the solution containing the linker molecules, and the values according to the process of the constituents of the solution containing the small molecule drug and the constituents of the fluid obtained by the mixing in the microreactor 101, such as the ratio of an unbound substance or the generation ratios of a plurality of bound substances.

The data obtained at the analyzing section 109 is sent to the operation/analysis section 108 through the signal line 113. By solving the inverse problem so as to reproduce the obtained data, process rate equations including the process rate constants for the linker molecule binding reaction process and process rate equations including the process rate constants for the small molecule drug binding reaction process in the bioconjugation reaction process are computed. Time series data of the generation efficiency of the drug conjugate is calculated from the determined process rate constants, and optimum process conditions that maximize the generation efficiency of the target drug conjugate are predicted.

The process conditions that are predicted to maximize the generation efficiency of the target drug conjugate are used as the next experimental conditions, and the next experimental conditions are displayed, as a change instruction, on the operation/analysis section 108. According to the displayed change instruction, the system configuration on the latter stage side of the first liquid delivery pump 105, the second liquid delivery pump 106, and the third liquid delivery pump 403 is changed. Examples of items of the change instruction displayed on the operation/analysis section 108 include: the flow rates of the solution containing the bio-related substance (first solution), the solution containing the linker molecules (second solution), and the solution containing the small molecule drug (third solution); the temperatures of the temperature adjusting apparatuses 115, 116, 117, 118, 406, and 407; the internal diameter and length of the residence section 112; the internal diameter and length of the residence section 405; the concentrations of the bio-related substance, the linker molecules, and the small molecule drug; the solvents of the solution containing the bio-related substance, the solution containing the linker molecules, and the solution containing the small molecule drug; and the like, and some of these.

### Specific Example 6

### Condition Optimization of Extension Reaction Process

In the condition-exploration microreactor system 4, a microreactor is used for mixing the solution containing the recipient substance and the solution containing the additive substance. Furthermore, a microreactor is used also for mixing the obtained fluid and the solution containing the additive substance. Here, the solution containing the additive substance mixed with the solution containing the recipient substance and the solution containing the additive substance mixed with the obtained fluid may be identical solutions, may have different concentrations, can have different types of solvent, or can have different types of additive substance.

In the first microreactor, the solution containing the recipient substance and the solution containing the additive substance are introduced into the micro flow paths which are micro reaction fields, and the extension reaction process is initiated by mixing them in the micro flow paths. Furthermore, in the second microreactor, the obtained fluid and the solution containing the additive substance are introduced into the micro flow paths which are micro reaction fields, and the extension reaction process is progressed by mixing them in the micro flow paths.

By using the microreactors, the flow rate ratios between the solution containing the recipient substance and the two types of the solutions containing the additive substances, the extension reaction process initiation timing, and the extension reaction process end timing can be controlled strictly. Accordingly, precise control of the mixing ratio between the recipient substance which is a constituent of the solution containing the recipient substance and the additive substance which is a constituent of the solution containing the additive substance, and precise control of the process time are possible. Accordingly, it is possible to generate a substance with the additive substance added at desired positions of the recipient substance with a high yield.

In the condition-exploration microreactor system 4, the operation/analysis section 108 constructs an extension reaction process model including process rate equations including process rate constants for reactions that generate the target substances and process rate equations including process rate constants for side reactions.

The solution containing the recipient substance is prepared in the first raw material container 102. The solution containing the additive substance is prepared in the second raw material container 103. The solution containing the additive substance is prepared in the third raw material container 402.

First, the solution prepared in the first raw material container 102 and containing the recipient substance is delivered from the first raw material container 102 to the first inlet of the microreactor 101 by the first liquid delivery pump 105. In addition, the solution prepared in the second raw material container 103 and containing the additive substance is delivered from the second raw material container 103 to the second inlet of the microreactor 101 by the second pump 106. Furthermore, the solution prepared in the third raw material container 402 and containing the additive substance is delivered from the third raw material container 402 to the first inlet of the microreactor 401 by the third liquid delivery pump 403.

Next, the solution containing the recipient substance and the solution containing the additive substance are mixed in the microreactor 101. By the mixing, the extension reaction process of the solution containing the recipient substance and the solution containing the additive substance is initiated. The extension reaction process further progresses while a product fluid generated in the microreactor 101 flows in the subsequent residence section 112 toward downstream.

Next, the solution containing the additive substance and the fluid obtained by the mixing in the microreactor 101 are mixed in the microreactor 401. By the mixing, the extension reaction process of the solution containing the additive substance and the fluid obtained by the mixing in the microreactor 101 is initiated. The extension reaction process further progresses while the product fluid generated in the microreactor 101 flows in the subsequent residence section 405 toward downstream.

Next, the product fluid discharged from the microreactor 401 and the subsequent residence section 405 is collected into the product collection container 104. The process is initiated in the micro flow paths of the microreactor 101, proceeds through the subsequent residence section 112, proceeds through the micro flow paths of the microreactor 401, and proceeds through the subsequent residence section 405, and then a product fluid which is a solution containing the substance to which the additive substance has been added is obtained.

An amount of the product fluid collected in the product collection container 104 that is required for performing an analysis in the analyzing section 109 is delivered to the analyzing section 109 by the sampling for analysis 114. The analyzing section 109 measures the constituents of the solution containing the recipient substance, the constituents of the two types of the solutions containing the additive substances, the values according to the process of the constituents of the solution containing the recipient substance and the constituents of the solution containing the additive substance, and the values according to the process of the constituents of the solution containing the additive substance and the constituents of the fluid obtained by the mixing in the microreactor 101, such as the ratio of substances that have not been added and the generation ratios of a plurality of added substances.

The data obtained at the analyzing section 109 is sent to the operation/analysis section 108 through the signal line 113. By solving the inverse problem so as to reproduce the obtained data, process rate equations including the process rate constants for reaction processes that generate the target products and process rate equations including the process rate constants for side reaction processes are computed. Time series data of the generation efficiency of the added substance is calculated from the determined process rate constants, and optimum process conditions that maximize the generation efficiency of the target added substance are predicted.

The process conditions that are predicted to maximize the generation efficiency of the target added substance are used as the next experimental conditions, and the next experimental conditions are displayed, as a change instruction, on the operation/analysis section 108. According to the displayed change instruction, the system configuration on the latter stage side of the first liquid delivery pump 105, the second liquid delivery pump 106, and the third liquid delivery pump 403 is changed. Examples of items of the change instruction displayed on the operation/analysis section 108 include: the flow rates of the solution containing the recipient substance (first solution), the solution containing the additive substance (second solution), and the solution containing the additive substance (third solution); the temperatures of the temperature adjusting apparatuses 115, 116, 117, 118, 406, and 407; the internal diameter and length of the residence section 112; the internal diameter and length of the residence section 405; the concentrations of the recipient substance and the two types of the additive substances; the solvents of the solution containing the recipient substance and the two types of the solutions containing the additive substances; and the like, and some of these.

The condition-exploration microreactor system 4 and the process condition optimization method using the same mentioned above make it possible to reduce the number of experiments required for the optimization of process conditions such as reactions as much as possible, to predict values for factors that have not been experimented or factors that cannot be measured, and to reduce the time required for experiments and the amount of reagents (waste) required as much as possible.

### Third Embodiment

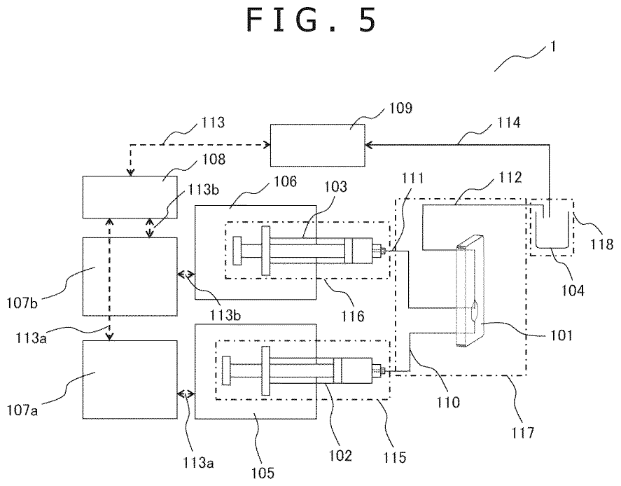

The condition-exploration microreactor system according to a third embodiment of the present invention is explained with reference to FIG. 5.

FIG. 5 is a schematic diagram of the condition-exploration microreactor system 1 according to the present embodiment, and is equivalent to a modification example of the first embodiment (FIG. 1).

As depicted in FIG. 5, the condition-exploration microreactor system 1 according to the present embodiment includes a plurality of the control sections 107 (here, two control sections 107a and 107b), and is configured such that the plurality of control sections 107a and 107b individually operate the liquid delivery pumps 105 and 106.

The control section 107a is connected with the operation/analysis section 108 and the first liquid delivery pump 105 by the signal lines 113a. In addition, the control section 107b is connected with the operation/analysis section 108 and the second liquid delivery pump 106 by the signal lines 113b.

By adopting the configuration as in the present embodiment in which a control section 107 and a liquid delivery pump are provided one-to-one, it is sufficient if combinations of control sections and liquid delivery pumps are added according to the number of microreactors in the system in a case where the number of the microreactors is increased, and the scalability of the system is enhanced.

### Fourth Embodiment

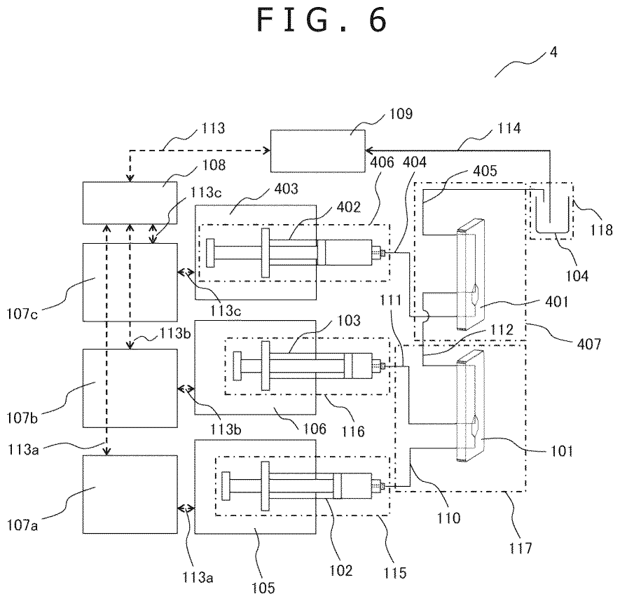

The condition-exploration microreactor system according to a fourth embodiment of the present invention is explained with reference to FIG. 6.

FIG. 6 is a schematic diagram of the condition-exploration microreactor system 4 according to the present embodiment, and is equivalent to a modification example of the second embodiment (FIG. 4).

As depicted in FIG. 6, the condition-exploration microreactor system 4 according to the present embodiment includes a plurality of the control sections 107 (here, three control sections 107a, 107b, and 107c), and is configured such that the plurality of control sections 107a, 107b, and 107c individually operate the liquid delivery pumps 105, 106, and 403.

The control section 107a is connected with the operation/analysis section 108 and the first liquid delivery pump 105 by the signal lines 113a. In addition, the control section 107b is connected with the operation/analysis section 108 and the second liquid delivery pump 106 by the signal lines 113b. In addition, the control section 107c is connected with the operation/analysis section 108 and the third liquid delivery pump 403 by the signal lines 113c.

By adopting the configuration as in the present embodiment in which a control section 107 and a liquid delivery pump are provided one-to-one, it is sufficient if combinations of control sections and liquid delivery pumps are added according to the number of microreactors in the system in a case where the number of the microreactors is increased, and the scalability of the system is enhanced.

Note that the present invention is not limited to the embodiments described above, and includes various modification examples. For example, the embodiments described above are explained in detail in order to explain the present invention in an easy-to-understand manner, and the present invention is not necessarily limited to embodiments including all the constituent elements explained. In addition, some of the constituent elements of an embodiment can be replaced with constituent elements of another embodiment, and also constituent elements of an embodiment can also be added to the constituent elements of another embodiment. In addition, some of the constituent elements of each embodiment can additionally have other constituent elements, be deleted, or be replaced with other constituent elements.

For example, the condition-exploration microreactor systems may have configurations in which three or more microreactors are connected in series. The configuration of the condition-exploration microreactor system 1 or the condition-exploration microreactor system 4 described above can also be directly connected repetitively for each type of process. An n-th (n is an integer equal to or greater than two) microreactor that mixes a mixed fluid generated in the former stage microreactor and a mixed fluid prepared in a container can be connected in series on the latter stage side of the first microreactor, and a product collection container can be provided last.

### REFERENCE SIGNS LIST

1: Condition-exploration microreactor system
101: Microreactor
102: First raw material container
103: Second raw material container
104: Product collection container
105: First liquid delivery pump
106: Second liquid delivery pump
107, 107a, 107b, 107c: Control section
108: Operation/analysis section
109: Analyzing section
110: First connecting section
111: Second connecting section
112: Residence section
113, 113a, 113b, 113c: Signal line
114: Sampling for analysis
115, 116, 117, 118: Temperature adjusting apparatus
2: Microreactor
201: Upper plate
202: Lower plate
203: High-flow-rate side flow path (micro flow path)
203a, 203b: Branch flow path
204: Low-flow-rate side flow path (micro flow path)
205: Mixing flow path (micro flow path)
206: Confluence
207: High-flow-rate side fluid inlet (through-hole)
208: Low-flow-rate side fluid inlet (through-hole)
209: Fluid outlet (through-hole)
4: Condition-exploration microreactor system
401: Microreactor
402: Third raw material container
403: Third liquid delivery pump
404: Third connecting section
405: Residence section
406, 407: Temperature adjusting apparatus

1. A condition-exploration microreactor system comprising:
a microreactor having two inlets from which fluids are introduced and a flow path where the fluids are caused to merge, the microreactor mixing, in the flow path, a first raw material introduced from a first inlet of the two inlets and a second raw material introduced from a second inlet of the two inlets;
a first raw material container in which the first raw material is prepared;
a second raw material container in which the second raw material is prepared;
a product collection container that collects a product obtained by mixing the first raw material and the second raw material in the microreactor;
a first liquid delivery pump that delivers the first raw material in the first raw material container to the first inlet;
a second liquid delivery pump that delivers the second raw material in the second raw material container to the second inlet;
a control section that operates the first liquid delivery pump and the second liquid delivery pump;
an analyzing section that analyzes the product; and
an operation/analysis section that receives an input of a signal from and outputs a signal to the control section and the analyzing section, and analyzes optimization of a process condition, wherein
a process model for a process in which the product is generated from the first raw material and the second raw material is constructed,
a process rate constant in the process model is calculated, and
a next experimental condition is decided on a basis of time-series data calculated from the process rate constant.

2. The condition-exploration microreactor system according to claim 1, wherein
a system configuration on a latter stage side of the first liquid delivery pump and the second liquid delivery pump is changed according to a change instruction displayed on the operation/analysis section.

3. The condition-exploration microreactor system according to claim 1, wherein
the product is a nanoparticle internally encapsulating a substance.

4. The condition-exploration microreactor system according to claim 1, wherein
the product is a chemically-modified product.

5. The condition-exploration microreactor system according to claim 1, wherein
the product has a repetitive structure formed through condensation.

6. The condition-exploration microreactor system according to claim 1, wherein
the condition-exploration microreactor system includes a plurality of the control sections, and
the plurality of control sections individually operate respective liquid delivery pumps.

7. A process condition optimization method of optimizing a process condition of a microreactor comprising:
(a) a step of constructing a process model for a process in which a product is generated from a first raw material and a second raw material;
(b) a step of implementing the process in which the product is generated from the first raw material and the second raw material under a predetermined condition;
(c) a step of analyzing the product generated at the step (b), and acquiring experiment data;
(d) a step of calculating a process rate constant in the process model constructed at the step (a) by using the experiment data acquired at the step (c);
(e) a step of calculating time-series data from the process rate constant calculated at the step (d); and
(f) a step of deciding a next experimental condition on a basis of the time series data calculated at the step (e).

8. The process condition optimization method according to claim 7, wherein
the process condition optimization method further includes (g) a step of displaying a change instruction on a basis of the experimental condition decided at the step (f), and
a system configuration in which the microreactor is incorporated is changed according to the change instruction.

9. The process condition optimization method according to claim 7, wherein
the product is a nanoparticle internally encapsulating a substance.

10. The process condition optimization method according to claim 7, wherein
the product is a chemically-modified product.

11. The process condition optimization method according to claim 7, wherein
the product has a repetitive structure formed through condensation.
